(19) United States
(12) Patent Application Publication
(10) Pub. No.: US 2009/0176996 A1
(43) Pub. Date: Jul. 9, 2009

(54) PROCESS FOR THE PREPARATION OF SULFAMIDE DERIVATIVES

(76) Inventors: Ahmed Abdel-Magid, Ambler, PA (US); Steven J Mehrman, Quakertown, PA (US); Caterina Ferraro, Fresh Meadows, NY (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/349,585

(22) Filed: Jan. 7, 2009

Related U.S. Application Data
(60) Provisional application No. 61/019,445, filed on Jan. 7, 2008.

(51) Int. Cl. C07D 333/58; C07C 303/34

(52) U.S. Cl. 549/58; 564/79

(57) ABSTRACT

The present invention is directed to novel processes for the preparation of sulfamide derivatves, useful in the treatment of epilepsy and related disorders.

### CROSS REFERENCE TO RELATED APPLICATIONS

This application claims the benefit of U.S. Provisional Application 61/019,445, filed on Jan. 7, 2008, which is incorporated by reference herein in its entirety.

### FIELD OF THE INVENTION

The present invention is directed to novel processes for the preparation of sulfamide derivatves, useful in the treatment of epilepsy and related disorders.

### BACKGROUND OF THE INVENTION

Epilepsy describes a condition in which a person has recurrent seizures due to a chronic, underlying process. Epilepsy refers to a clinical phenomenon rather than a single disease entity, since there are many forms and causes of epilepsy. Using a definition of epilepsy as two or more unprovoked seizures, the incidence of epilepsy is estimated at approximately 0.3 to 0.5 percent in different populations throughout the world, with the prevalence of epilepsy estimated at 5 to 10 people per 1000.

An essential step in the evaluation and management of a patient with a seizure is to determine the type of seizure that has occurred. The main characteristic that distinguishes the different categories of seizures is whether the seizure activity is partial (synonymous with focal) or generalized.

Partial seizures are those in which the seizure activity is restricted to discrete areas of the cerebral cortex. If consciousness is fully preserved during the seizure, the clinical manifestations are considered relatively simple and the seizure is termed a simple-partial seizure. If consciousness is impaired, the seizure is termed a complex-partial seizure. An important additional subgroup comprises those seizures that begin as partial seizures and then spread diffusely throughout the cortex, which are known as partial seizures with secondary generalization.

Generalized seizures involve diffuse regions of the brain simultaneously in a bilaterally symmetric fashion. Absence or petit mal seizures are characterized by sudden, brief lapses of consciousness without loss of postural control. Atypical absence seizures typically include a longer duration in the lapse of consciousness, less abrupt onset and cessation, and more obvious motor signs that may include focal or lateralizing features. Generalized Tonic-clonic or grand mal seizures, the main type of generalized seizures, are characterized by abrupt onset, without warning. The initial phase of the seizure is usually tonic contraction of muscles, impaired respiration, a marked enhancement of sympathetic tone leading to increased heart rate, blood pressure, and pupillary size. After 10-20 s, the tonic phase of the seizure typically evolves into the clonic phase, produced by the superimposition of periods of muscle relaxation on the tonic muscle contraction. The periods of relaxation progressively increase until the end of the ictal phase, which usually lasts no more than 1 min. The postictal phase is characterized by unresponsiveness, muscular flaccidity, and excessive salivation that can cause stridorous breathing and partial airway obstruction. Atonic seizures are characterized by sudden loss of postural muscle tone lasting 1-2 s. Consciousness is briefly impaired, but there is usually no postictal confusion. Myoclonic seizures are characterized by a sudden and brief muscle contraction that may involve one part of the body or the entire body.

Parker, M. H., et al., in US Patent Publication US2006/0047001 A1, published Mar. 2, 2006 disclose sulfamide derivatives useful in the treatment of epilepsy and related disorders and a process for their preparation. There remains a need for a process suitable for the preparation of large scale material and/or for commercial preparation of the sulfamide derivative compounds.

### SUMMARY OF THE INVENTION

The present invention is further directed to a process for the preparation of compounds of formula (I-A)

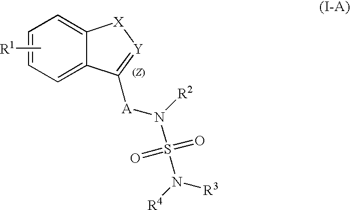

wherein

R¹ is selected from the group consisting of hydrogen, halogen, hydroxy, methoxy, trifluoromethyl, nitro and cyano;

X—Y is selected from the group consisting of —S—CH—, —S—C(CH₃)—, —O—CH—, —O—C(CH₃)—, —N(CH₃)—CH— and —CH═CH—CH—;

A is selected from the group consisting of —CH₂— and —CH(CH₃)—;

R² is hydrogen;

R³ and R⁴ are each independently selected from the group consisting of hydrogen and C₁₋₄alkyl;

alternatively, R³ and R⁴ are taken together with the nitrogen atom to which they are bound to form a 5 to 7 membered, saturated, partially unsaturated or aromatic ring structure, optionally containing one to three additional heteroatoms independently selected from the group consisting of O, N and S;

or a pharmaceutically acceptable salt thereof; comprising

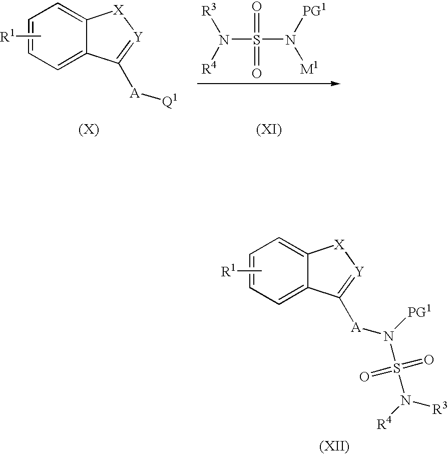

reacting a compound of formula (X), wherein Q¹ is a leaving group with a compound of formula (XI), wherein PG¹ is hydrogen or a nitrogen protecting group, and wherein M¹ is hydrogen; in the presence of a base; in an organic solvent; to yield the corresponding compound of formula (XII);

or reacting a compound of formula (X), wherein Q¹ is a leaving group with a compound of formula (XI), wherein PG¹ is a nitrogen protecting group, and wherein M¹ is a metal cation or a tertiary ammonium ion; in an organic solvent; to yield the corresponding compound of formula (XII);

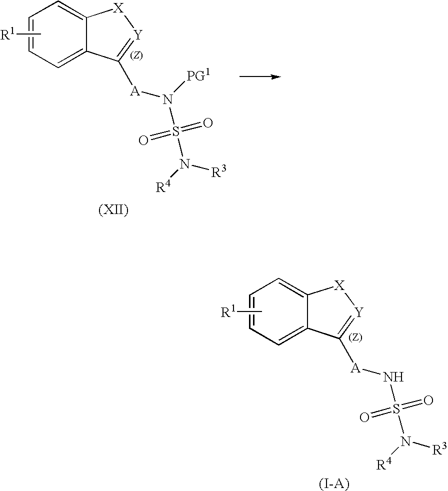

de-protecting the compound of formula (XII), to yield the corresponding compound of formula (I-A).

In an embodiment, the present invention is directed to a process for the preparation of a compound of formula (I-S)

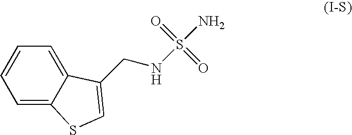

also known as N-(benzo[b]thien-3-ylmethyl)-sulfamide, or a pharmaceutically acceptable salt thereof; comprising

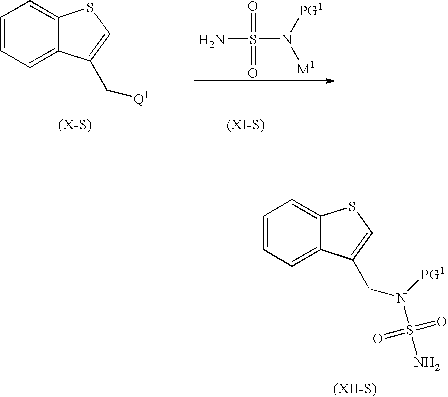

reacting a compound of formula (X-S), wherein Q¹ is a leaving group with a compound of formula (XI-S), wherein PG¹ is hydrogen or a nitrogen protecting group, and wherein M¹ is hydrogen; in the presence of a base; in an organic solvent; to yield the corresponding compound of formula (XII-S);

or reacting a compound of formula (X-S), wherein Q¹ is a leaving group with a compound of formula (XI-S), wherein PG¹ is a nitrogen protecting group, and wherein M¹ is a metal cation or a tertiary ammonium ion; in an organic solvent; to yield the corresponding compound of formula (XII-S);

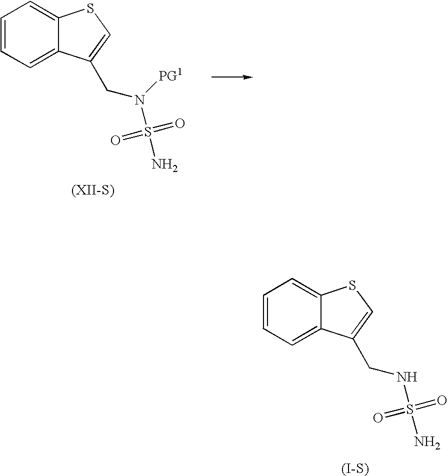

de-protecting the compound of formula (XII-S), to yield the corresponding compound of formula (I-S).

The present invention is further directed to a process for the preparation of a compound of formula (II-A)

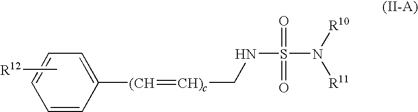

wherein

R¹² is selected from the group consisting of hydrogen, halogen, hydroxy, methoxy, trifluoromethyl, nitro and cyano;

c is an integer from 0 to 2;

R¹⁰ and R¹¹ are each independently selected from the group consisting of hydrogen and C₁₋₄alkyl;

alternatively, R¹⁰ and R¹¹ are taken together with the nitrogen atom to which they are bound to form a 5 to 7 membered, saturated, partially unsaturated or aromatic ring structure, optionally containing one to three additional heteroatoms independently selected from the group consisting of O, N and S;

or a pharmaceutically acceptable salt thereof; comprising

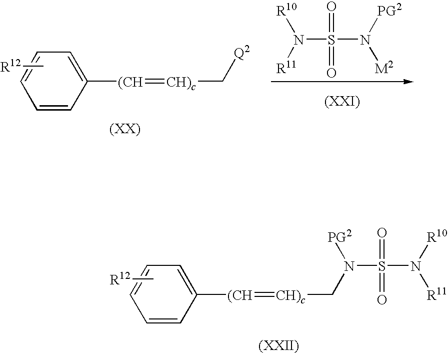

reacting a compound of formula (XX), wherein Q² is a leaving group with a compound of formula (XXI), wherein PG² is hydrogen or a nitrogen protecting group, and wherein M² is hydrogen; in the presence of a base; in an organic solvent; to yield the corresponding compound of formula (XXII);

or reacting a compound of formula (XX), wherein Q² is a leaving group with a compound of formula (XXI), wherein PG² is a nitrogen protecting group, and wherein M¹ is a metal cation or a tertiary ammonium ion; in an organic solvent; to yield the corresponding compound of formula (XXII);

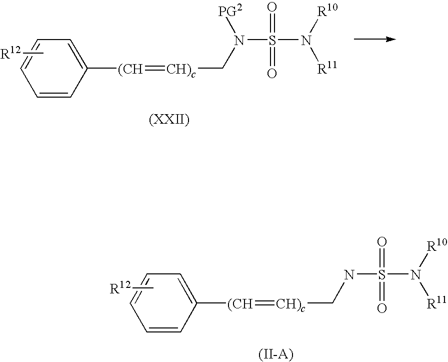

de-protecting the compound of formula (XXII), to yield the corresponding compound of formula (II-A).

The present invention is further directed to compounds of formula (XII)

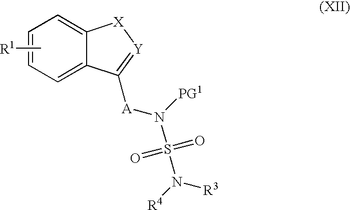

wherein

PG¹ is hydrogen or a nitrogen protecting group (preferably, PG¹ is t-butoxycarbonyl);

R¹ is selected from the group consisting of hydrogen, halogen, hydroxy, methoxy, trifluoromethyl, nitro and cyano;

X—Y is selected from the group consisting of —S—CH—, —S—C(CH₃)—, —O—CH—, —O—C(CH₃)—, —N(CH₃)—CH— and —CH═CH—CH—;

A is selected from the group consisting of —CH₂— and —CH(CH₃)—;

R² is hydrogen;

R³ and R⁴ are each independently selected from the group consisting of hydrogen and C₁₋₄alkyl;

alternatively, R³ and R⁴ are taken together with the nitrogen atom to which they are bound to form a 5 to 7 membered, saturated, partially unsaturated or aromatic ring structure, optionally containing one to three additional heteroatoms independently selected from the group consisting of O, N and S.

In an embodiment, the present invention is directed to compounds of formula (XII-S)

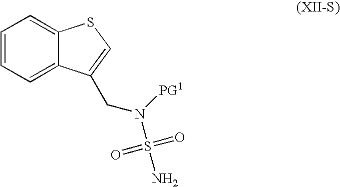

wherein PG¹ is hydrogen or a nitrogen protecting group (preferably, PG¹ is t-butoxycarbonyl).

The present invention is further directed to compounds of formula (XXII)

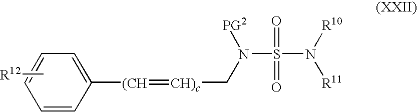

wherein

PG² is hydrogen or a nitrogen protecting group (preferably, PG¹ is t-butoxycarbonyl);

R¹² is selected from the group consisting of hydrogen, halogen, hydroxy, methoxy, trifluoromethyl, nitro and cyano;

c is an integer from 0 to 2;

R¹⁰ and R¹¹ are each independently selected from the group consisting of hydrogen and C₁₋₄alkyl;

alternatively, R¹⁰ and R¹¹ are taken together with the nitrogen atom to which they are bound to form a 5 to 7 membered, saturated, partially unsaturated or aromatic ring structure, optionally containing one to three additional heteroatoms independently selected from the group consisting of O, N and S.

The present invention is further directed to compounds of formula (II-A)

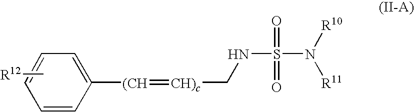

wherein

R¹² is selected from the group consisting of hydrogen, halogen, hydroxy, methoxy, trifluoromethyl, nitro and cyano;

c is an integer from 0 to 2;

R¹⁰ and R¹¹ are each independently selected from the group consisting of hydrogen and C₁₋₄alkyl;

alternatively, R¹⁰ and R¹¹ are taken together with the nitrogen atom to which they are bound to form a 5 to 7 membered, saturated, partially unsaturated or aromatic ring structure, optionally containing one to three additional heteroatoms independently selected from the group consisting of O, N and S;

provided that when c is 0; then R¹² is other than hydrogen;

and pharmaceutically acceptable salts thereof. The compounds of formula (II-A) are useful for the treatment of epilepsy and related disorders.

The present invention is further directed to a product prepared according to the process described herein.

Illustrative of the invention is a pharmaceutical composition comprising a pharmaceutically acceptable carrier and the product prepared according to the process described herein. An illustration of the invention is a pharmaceutical composition made by mixing the product prepared according to the process described herein and a pharmaceutically acceptable carrier. Illustrating the invention is a process for making a pharmaceutical composition comprising mixing the product prepared according to the process described herein and a pharmaceutically acceptable carrier.

Exemplifying the invention are methods of treating a epilepsy or a related disorder comprising administering to a subject in need thereof a therapeutically effective amount of any of the compounds or pharmaceutical compositions described above.

Another example of the invention is the use of any of the compounds described herein in the preparation of a medicament for treating epilepsy or a related disorder in a subject in need thereof.

### DETAILED DESCRIPTION OF THE INVENTION

The present invention is directed to a process for the preparation of compounds of formula (I-A) and compounds of formula (I-A)

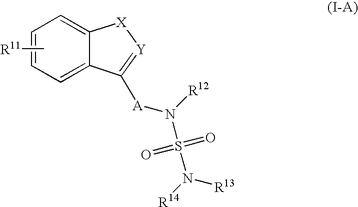

wherein all substituent groups are as herein defined, and pharmaceutically acceptable salts thereof. The compounds of the present invention are useful in the treatment of epilepsy and related disorders.

The present invention is further directed to a process for the preparation of compounds of formula (II-A)

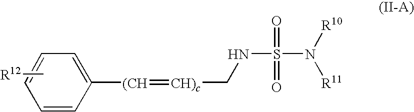

wherein all substituent groups are as herein defined, and pharmaceutically acceptable salts thereof. The present invention is further directed to compounds of formula (II-A) wherein all substituent groups are as herein defined; and pharmaceutically acceptable salts thereof. The compounds of the present invention are useful in the treatment of epilepsy and related disorders.

The present invention is further directed to compounds of formula (XII)

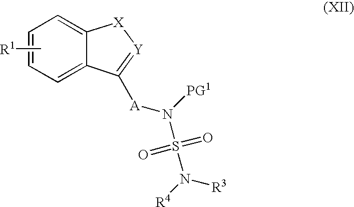

wherein all substituent groups are as herein defined. The compounds of formula (XII) are useful as intermediates in the synthesis of the compounds of formula (I-A). In an embodiment, the present invention is directed to compounds of formula (XII-S)

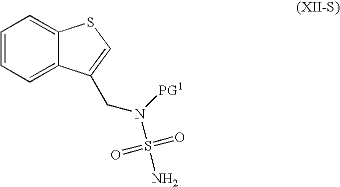

wherein all substituent groups are as herein defined; useful as intermediates in the synthesis of the compound of formula (I-S).

The present invention is further directed to compounds of formula (XXII)

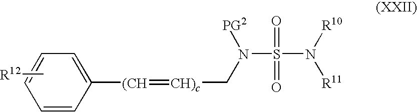

wherein all substituent groups are as herein defined. The compounds of formula (XXII) are useful as intermediates in the synthesis of the compounds of formula (II-A).

As used herein, unless otherwise noted, the terms “epilepsy and related disorders” or “epilepsy or related disorder” shall mean any disorder in which a subject (preferably a human adult, child or infant) experiences one or more seizures and/or tremors. Suitable examples include, but are not limited to, epilepsy (including, but not limited to, localization-related epilepsies, generalized epilepsies, epilepsies with both generalized and local seizures, and the like), seizures as a complication of a disease or condition (such as seizures associated with encephalopathy, phenylketonuria, juvenile Gaucher's disease, Lundborg's progressive myoclonic epilepsy, stroke, head trauma, stress, hormonal changes, drug use or withdrawal, alcohol use or withdrawal, sleep deprivation, and the like), essential tremor, restless limb syndrome, and the like. Preferably, the disorder is selected from epilepsy (regardless of type, underlying cause or origin), essential tremor or restless limb syndrome, more preferably, the disorder is epilepsy (regardless of type, underlying cause or origin) or essential tremor.

In an embodiment of the present invention, PG¹ is hydrogen or a nitrogen protecting group. In another embodiment of the present invention, PG¹ is a nitrogen protecting group. In another embodiment of the present invention, PG¹ is hydrogen, BOC or Cbz. In another embodiment of the present invention, PG¹ is BOC or Cbz. In another embodiment of the present invention PG¹ is hydrogen or BOC. In an embodiment of the present invention, PG¹ is BOC.

In an embodiment of the present invention, PG² is hydrogen or a nitrogen protecting group. In another embodiment of the present invention, PG² is a nitrogen protecting group. In another embodiment of the present invention, PG² is hydrogen, BOC or Cbz. In another embodiment of the present invention, PG² is BOC or Cbz. In another embodiment of the present invention PG² is hydrogen or BOC. In an embodiment of the present invention, PG² is BOC.

In an embodiment of the present invention, in the compound of formula (II-A), c is 0. In another embodiment of the present invention, in the compound of formula (II-A), c is 1. In another embodiment of the present invention, in the compound of formula (II-A), R¹² is hydrogen, c is an integer from 0 to 1, R¹⁰ is hydrogen and R¹¹ is hydrogen.

In an embodiment, the present invention is directed to a compound of formula (I)

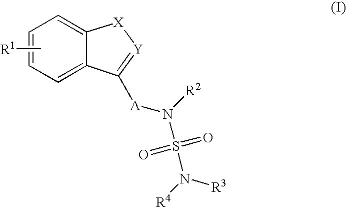

wherein R¹ is selected from the group consisting of hydrogen, halogen, hydroxy, methoxy, trifluoromethyl, nitro and cyano; X—Y is selected from the group consisting of —S—CH—, —S—C(CH₃)—, —O—CH—, —O—C(CH₃)—, —N(CH₃)—CH— and —CH═CH—CH—; A is selected from the group consisting of —CH₂— and —CH(CH₃)—; R² is hydrogen; R³ and R⁴ are each independently selected from the group consisting of hydrogen and methyl; alternatively, R³ and R⁴ are taken together with the nitrogen atom to which they are bound to form a 5 to 7 membered, saturated, partially unsaturated or aromatic ring structure, optionally containing one to two additional heteroatoms independently selected from the group consisting of O, N and S; or a pharmaceutically acceptable salt thereof.

In another embodiment of the present invention are compounds of formula (I) wherein R¹ is selected from the group consisting of hydrogen and halogen; X—Y is selected from the group consisting of —S—CH—, —S—C(CH₃)—, —O—CH—, —O—C(CH₃)—, —N(CH₃)—CH— and —CH═CH—CH—; A is selected from the group consisting of —CH₂— and —CH(CH₃)—; R² is hydrogen; R³ and R⁴ are each independently selected from the group consisting of hydrogen and methyl; and pharmaceutically acceptable salts thereof.

In another embodiment of the present invention are compounds of formula (I) wherein R¹ is selected from the group consisting of hydrogen and halogen; wherein the halogen is bound at the 4-, 5- or 7-position; X—Y is selected from the groups consisting of —O—CH—, —O—C(CH₃)—, —S—CH—, —S—C(CH₃)—, —N(CH₃)—CH— and —CH═CH—CH—; A is selected from the group consisting of —CH₂— and —CH(CH₃)—; R² is hydrogen; R³ and R⁴ are each hydrogen; and pharmaceutically acceptable salts thereof.

In another embodiment of the present invention are compounds of formula (I) wherein R¹ is hydrogen; X—Y is selected from the groups consisting of —O—CH—, —O—C(CH₃)—, —S—CH—, —S—C(CH₃)—, —N(CH₃)—CH— and —CH═CH—CH—; A is selected from the group consisting of —CH₂— and —CH(CH₃)—; R² is hydrogen; R³ and R⁴ are each hydrogen; and pharmaceutically acceptable salts thereof.

In another embodiment of the present invention are compounds of formula (I) wherein R¹ is selected from the group consisting of hydrogen halogen, hydroxy, methoxy, trifluoromethyl, nitro and cyano; preferably, R¹ is selected from the group consisting of hydrogen and halogen; more preferably, R¹ is selected from the group consisting of hydrogen and halogen, wherein the halogen is bound at the 4-, 5- or 7-position; X—Y is —S—CH—; A is selected from the group consisting of —CH₂— and —CH(CH₃)—; R² is hydrogen; R³ and R⁴ are each independently selected from the group consisting of hydrogen and halogen; preferably, R³ and R⁴ are each hydrogen; and pharmaceutically acceptable salts thereof.

In an embodiment of the present invention R¹ is selected from the group consisting of hydrogen, chloro, fluoro and bromo. In another embodiment of the present invention, the R¹ group is other than hydrogen and bound at the 4-, 5- or 7-position, preferably at the 5-position. In yet another embodiment of the present invention, the R¹ group is other than hydrogen and bound at the 5-, 6- or 8-position, preferably at the 6-position. In yet another embodiment of the present invention, R¹ is selected from the group consisting of hydrogen and halogen. In yet another embodiment of the present invention, R¹ is selected from the group consisting of hydroxy and methoxy. In yet another embodiment of the present invention, R¹ is selected from the group consisting of hydrogen, halogen and trifluoromethyl. In yet another embodiment of the present invention, R¹ is selected from the group consisting of hydrogen, halogen, trifluoromethyl, cyano and nitro. In yet another embodiment of the present invention, R¹ is selected from the group consisting of hydrogen, halogen, trifluoromethyl and cyano. In yet another embodiment of the present invention, R¹ is selected from the group consisting of trifluoromethyl and cyano. In yet another embodiment of the present invention, R1 is selected from the group consisting of hydrogen, 4-bromo, 5-chloro, 5-fluoro, 5-bromo, 5-trifluoromethyl-5-cyano and 7-cyano.

In an embodiment of the present invention R² is hydrogen. In another embodiment of the present invention R³ and R⁴ are each hydrogen. In yet another embodiment of the present invention R² is hydrogen, R³ is hydrogen and R⁴ is hydrogen.

In an embodiment of the present invention, R³ and R⁴ are each independently selected from the group consisting of hydrogen and C₁₋₄alkyl. In another embodiment of the present invention, R³ and R⁴ are taken together with the nitrogen atom to which they are bound to form a 5 to 7 membered, saturated, partially unsaturated or aromatic ring structure, optionally containing one to two additional heteroatoms independently selected from the group consisting of O, N and S.

In an embodiment of the present invention, R³ and R⁴ are each independently selected from the group consisting of hydrogen, methyl and ethyl. In another embodiment of the present invention, R³ and R⁴ are each independently selected from the group consisting of hydrogen and methyl. In yet another embodiment of the present invention, R³ and R⁴ are each independently selected from the group consisting of hydrogen and ethyl. In yet another embodiment of the present invention, R³is hydrogen and R⁴ is ethyl.

In an embodiment of the present invention R³ and R⁴ are taken together with the nitrogen atom to which they are bound to form a 5 to 7 membered, saturated, partially unsaturated or aromatic ring structure, optionally containing one to two additional heteroatoms independently selected from the group consisting of O, S and N. In another embodiment of the present invention R³ and R⁴ are taken together with the nitrogen atom to which they are bound to form a 5 to 7 membered saturated ring structure, optionally containing one to two additional heteroatoms independently selected from the group consisting of O, S and N. In another embodiment of the present invention R³ and R⁴ are taken together with the nitrogen atom to which they are bound to form a 5 to 7 membered aromatic ring structure, optionally containing one to two additional heteroatoms independently selected from the group consisting of O, S and N.

Preferably, R³ and R⁴ are taken together with the nitrogen atom to which they are bound to form a 5 to 6 membered saturated, partially unsaturated or aromatic ring structure, optionally containing one to two additional heteroatoms independently selected from the group consisting of O, S and N. More preferably, R³ and R⁴ are taken together with the nitrogen atom to which they are bound to form a 6 membered saturated, partially unsaturated or aromatic ring structure, optionally containing one to two additional heteroatoms independently selected from the group consisting of O, S and N.

Preferably, R³ and R⁴ are taken together with the nitrogen atom to which they are bound to form a 5 to 7 (more preferably 5 to 6) membered saturated or aromatic ring structure, optionally containing one to two (preferably one) additional heteroatoms independently selected from the group consisting of O, S and N (preferably O or N, more preferably N).

In another embodiment of the present invention, R³ and R⁴ are taken together with the nitrogen atom to which they are bound to form a 5 to 6 membered saturated or aromatic ring structure, optionally containing one to two (preferably one) additional heteroatoms independently selected from the group consisting of O, S and N (preferably O or N, more preferably, N).

Preferably, the 5 to 7 membered saturated, partially unsaturated or aromatic ring structure contains 0 to 1 additional heteroatoms independently selected from the group consisting of O, S and N. Preferably, the heteroatom is independently selected from the group consisting of O and N, more preferably, the heteroatom is N.

Suitable examples of the 5 to 7 membered, saturated, partially unsaturated or aromatic ring structures which optionally contain one to two additional heteroatoms independently selected from the group consisting of O, S and N include, but are not limited to pyrrolyl, pyrrolidinyl, pyrrolinyl, morpholinyl, piperidinyl, piperazinyl, imidazolyl, pyrazolyl, pyridyl, imidazolyl, thiomorpholinyl, pyrazinyl, triazinyl, azepinyl, and the like. Preferred 5 to 7 membered, saturated, partially unsaturated or aromatic ring structures which optional containing one to two additional heteroatoms independently selected from the group consisting of O, S and N include, but are not limited, to imidazolyl, pyrrolidinyl, piperidinyl and morpholinyl.

In an embodiment of the present invention A is —CH₂—.

In an embodiment of the present invention X—Y is selected from the group consisting of —S—CH—, —O—CH—, —O—C(CH₃)—, —N(CH₃)—CH— and —CH═CH—CH—. In another embodiment of the present invention X—Y is selected from the group consisting of —S—CH—, —O—CH—, —O—C(CH₃)— and —CH═CH—CH—. In yet another embodiment of the present invention X—Y is selected form the group consisting of —S—CH—, —O—CH—, —O—C(CH₃)— and —N(CH₃)—CH—. In yet another embodiment of the present invention X—Y is selected from the group consisting of —S—CH—, —O—CH—, —N(CH₃)—CH— and —CH═CH—CH—. In yet another embodiment of the present invention X—Y is selected from the group consisting of —S—CH—, —O—CH— and —CH═CH—C—. In yet another embodiment of the present invention, X—Y is selected from the group consisting of —S—CH— and —O—CH—. In yet another embodiment of the present invention, X—Y is selected from the group consisting of S—CH—, —S—C(CH₃)—, —O—CH—, —O—C(CH₃)— and —N(CH₃)—CH—.

In an embodiment of the present invention, X— is —S—CH—. In another embodiment of the present invention X—Y is —CH═CH═CH—. In yet another embodiment of the present invention X—Y is —N(CH₃)—CH—. In yet another embodiment of the present invention X—Y is selected from the group consisting of —O—CH— and —O—C(CH₃)—.

In an embodiment, the present invention is directed to a compounds selected from the group consisting of N-(benzo[b]thien-3-ylmethyl)-sulfamide; N-[(5-chlorobenzo[b]thien-3-yl)methyl]-sulfamide; N-(3-benzofuranylmethyl)-sulfamide; N-[(5-fluorobenzo[b]thien-3-yl)methyl]-sulfamide; N-(1-benzo[b]thien-3-ylethyl)-sulfamide; N-(1-naphthalenylmethyl)-sulfamide; N-[(2-methyl-3-benzofuranyl)methyl]-sulfamide; N-[(5-bromobenzo[b]thien-3-yl)methyl]-sulfamide; N-[(4-bromobenzo[b]thien-3-yl)methyl]-sulfamide; N-[(7-fluorobenzo[b]thien-3-yl)methyl]-sulfamide; N-[(1-methyl-1H-indol-3-yl)methyl]-sulfamide; N-[(4-trifluoromethylbenzo[b]thien-3-yl)methyl]-sulfamide; N-[(4-cyanobenzo[b]thien-3-yl)methyl]-sulfamoylpyrrolidine; N-[(benzo[b]thien-3-yl)methyl]-N′-ethylsulfamide; Imidazole-1-sulfonic acid [(benzo[b]thien-3-yl)methyl]-amide; and pharmaceutically acceptable salts thereof.

Additional embodiments of the present invention, include those wherein the substituents selected for one or more of the variables defined herein (i.e. R¹, R², R³, R⁴, X—Y and A) are independently selected to be any individual substituent or any subset of substituents selected from the complete list as defined herein. In another embodiment of the present invention is a process for the preparation of any single compound or subset of compounds selected from the representative compounds listed in Tables 1-2 below.

Unless otherwise noted, wherein a stereogenic center is present in a listed compound in the Tables 1-2 below, the compound was prepared as a mixture of stereo-configurations.

TABLE 1
Representative Compounds of Formula (I-A)

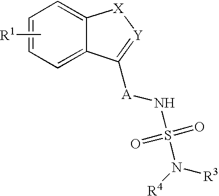

| ID No. | R¹ | -X-Y- | A | R³ | R⁴ |
|---|---|---|---|---|---|
| 1 | H | —S—CH— | —CH₂— | H | H |
| 3 | 5-Cl | —S—CH— | —CH₂— | H | H |
| 6 | H | —O—CH— | —CH₂— | H | H |
| 7 | H | —N(CH₃)—CH— | —CH₂— | H | H |
| 8 | 5-F | —S—CH— | —CH₂— | H | H |
| 9 | H | —S—CH— | —CH(CH₃)— | H | H |
| 10 | H | —CH═CH—CH— | —CH₂— | H | H |
| 13 | H | —O—C(CH₃) | —CH₂— | H | H |
| 15 | 5-Br | —S—CH— | —CH₂— | H | H |
| 17 | 4-Br | —S—CH— | —CH₂— | H | H |
| 18 | 7-F | —S—CH— | —CH₂— | H | H |
| 19 | 5-CF₃ | —S—CH— | —CH₂— | H | H |
| 20 | 5-CN | —S—CH— | —CH₂— | H | H |
| 21 | H | —S—CH— | —CH₂— | H | ethyl |

TABLE 2
Representative Compounds of Formula (I-A)

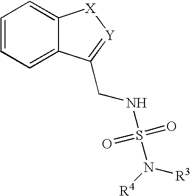

| ID | | R⁴ + R⁴ together |
|---|---|---|
| No. | -X-Y- | with the N atom |
| 101 | —S—CH— | N-pyrrolidinyl |
| 102 | —S—CH— | N-imidazolyl |

As used herein, “halogen” shall mean chlorine, bromine, fluorine and iodine.

As used herein, the term “alkyl” whether used alone or as part of a substituent group, include straight and branched chains. For example, alkyl radicals include methyl, ethyl, propyl, isopropyl, butyl, isobutyl, sec-butyl, t-butyl, pentyl and the like. Unless otherwise noted, “C₁₋₄alkyl” means a carbon chain composition of 1-4 carbon atoms.

When a particular group is “substituted” (e.g., alkkyl, cycloalkyl, aryl, heteroaryl, heterocycloalkyl, etc.), that group may have one or more substituents, preferably from one to five substituents, more preferably from one to three substituents, most preferably from one to two substituents, independently selected from the list of substituents.

With reference to substituents, the term “independently” means that when more than one of such substituents is possible, such substituents may be the same or different from each other.

As used herein, the notation “*” shall denote the presence of a stereogenic center.

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention. Preferably, wherein the compound is present as an enantiomer, the enantiomer is present at an enantiomeric excess of greater than or equal to about 80%, more preferably, at an enantiomeric excess of greater than or equal to about 90%, more preferably still, at an enantiomeric excess of greater than or equal to about 95%, more preferably still, at an enantiomeric excess of greater than or equal to about 98%, most preferably, at an enantiomeric excess of greater than or equal to about 99%. Similalry, wherein the compound is present as a diastereomer, the diastereomer is present at an diastereomeric excess of greater than or equal to about 80%, more preferably, at an diastereomeric excess of greater than or equal to about 90%, more preferably still, at an diastereomeric excess of greater than or equal to about 95%, more preferably still, at an diastereomeric excess of greater than or equal to about 98%, most preferably, at an diastereomeric excess of greater than or equal to about 99%.

Furthermore, some of the crystalline forms for the compounds of the present invention may exist as polymorphs and as such are intended to be included in the present invention. In addition, some of the compounds of the present invention may form solvates with water (i.e., hydrates) or common organic solvents, and such solvates are also intended to be encompassed within the scope of this invention.

Under standard nomenclature used throughout this disclosure, the terminal portion of the designated side chain is described first, followed by the adjacent functionality toward the point of attachment. Thus, for example, a “phenylalkylaminocarbonylalkyl” substituent refers to a group of the formula

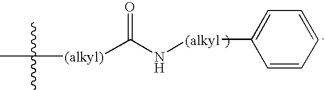

Abbreviations used in the specification, particularly the Schemes and Examples, are as follows:

| | |
|---|---|
| AcOH = | Acetic Acid |
| aq. = | Aqueous |
| conc. = | Concentrated |
| Cbz or CBz = | Benzyloxycarbonyl |
| DIPEA = | Diisopropylethylamine |
| DMF = | N,N-Dimethylformamide |
| DMSO = | Dimethylsulfoxide |
| Et₃N or TEA = | Triethylamine |
| EtOAc = | Ethyl Acetate |
| EtOH = | Ethanol |
| IPA = | Isopropyl Alcohol |
| MeOH = | Methanol |
| Mesylate = | Methane sulfonic acid anion (also known as |
| | methanesulfonate) |
| MTBE = | Methyl-tert-butyl Ether |
| NMM = | N-methylmorpholine (also known as 4- |
| | methylmorpholine) |
| satd. = | Saturated |
| t-BOC or Boc = | Tert-Butoxycarbonyl |
| TEA = | Triethylamine |
| TFA = | Trifluoroacetic Acid |
| TLC = | Thin Layer Chromatography |
| Tosylate = | p-Toluenesulfonic acid anion (p- |
| | toluenesulfonate) |
| Triflate = | Trifluoro-methanesulfonic acid anion (also |
| | known as trifluoromethanesulfonate) |

As used herein, unless otherwise noted, the term “isolated form” shall mean that the compound is present in a form which is separate from any solid mixture with another compound(s), solvent system or biological environment. In an embodiment, the present invention is directed to a process for the preparation of a compound of formula (I-A), preferably a compound of formula (I-S), in an isolated form.

As used herein, unless otherwise noted, the term “substantially pure compound” shall mean that the mole percent of impurities in the isolated compound is less than about 5 mole percent, preferably less than about 2 mole percent, more preferably, less than about 0.5 mole percent, most preferably, less than about 0.1 mole percent. In an embodiment, the present invention is directed to a process for the preparation of a compound of formula (I-A), preferably a compound of formula (I-S), as a substantially pure compound.

As used herein, unless otherwise noted, the term “substantially free of a corresponding salt form(s)” when used to described the compound of formula (I) shall mean that mole percent of the corresponding salt form(s) in the isolated base of formula (I) is less than about 5 mole percent, preferably less than about 2 mole percent, more preferably, less than about 0.5 mole percent, most preferably less than about 0.1 mole percent. In an embodiment, the present invention is directed to a process for the preparation of a compound of formula (I-A), preferably a compound of formula (I-S), as a compound substantially free of corresponding slat form(s).

The term “subject” as used herein, refers to an animal, preferably a mammal, most preferably a human, who has been the object of treatment, observation or experiment. Preferably, the subject has experienced and/or exhibited at least one symptom of the disease or disorder to be treated and/or prevented.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated.

As used herein, the term “composition” is intended to encompass a product comprising the specified ingredients in the specified amounts, as well as any product which results, directly or indirectly, from combinations of the specified ingredients in the specified amounts.

One skilled in the art will recognize that, where not otherwise specified, the reaction step(s) is performed under suitable conditions, according to known methods, to provide the desired product.

One skilled in the art will recognize that, in the specification and claims as presented herein, wherein a reagent or reagent class/type (e.g. base, solvent, etc.) is recited in more than one step of a process, the individual reagents are independently selected for each reaction step and may be the same of different from each other. For example wherein two steps of a process recite an organic or inorganic base as a reagent, the organic or inorganic base selected for the first step may be the same or different than the organic or inorganic base of the second step.

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

As used herein, unless otherwise noted, the term “aprotic solvent” shall mean any solvent that does not yield a proton. Suitable examples include, but are not limited to DMF, 1,4-dioxane, THF, acetonitrile, pyridine, dichloroethane, dichloromethane, MTBE, toluene, acetone, and the like.

As used herein, unless otherwise noted, the term “leaving group” shall mean a charged or uncharged atom or group which departs during a substitution or displacement reaction. Suitable examples include, but are not limited to, Br, Cl, I, mesylate, tosylate, triflate, and the like.

As used herein, unless otherwise noted, the term “nitrogen protecting group” shall mean a group which may be attached to a nitrogen atom to protect said nitrogen atom from participating in a reaction and which may be readily removed following the reaction. Suitable nitrogen protecting groups include, but are not limited to carbamates—groups of the formula —C(O)O—R wherein R is for example methyl, ethyl, t-butyl, benzyl, phenylethyl, CH₂═CH—CH₂—, and the like; amides—groups of the formula —C(O)—R′ wherein R′ is for example methyl, phenyl, trifluoromethyl, and the like; N-sulfonyl derivatives—groups of the formula —SO₂—R″ wherein R″ is for example tolyl, phenyl, trifluoromethyl, 2,2,5,7,8-pentamethylchroman-6-yl-, 2,3,6-trimethyl-4-methoxybenzene, and the like. Other suitable nitrogen protecting groups may be found in texts such as T. W. Greene & P. G. M. Wuts, Protective Groups in Organic Synthesis, John Wiley & Sons, 1991.

One skilled in the art will recognize that wherein a reaction step of the present invention may be carried out in a variety of solvents or solvent systems, said reaction step may also be carried out in a mixture of the suitable solvents or solvent systems.

Where the processes for the preparation of the compounds according to the invention give rise to mixture of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their component enantiomers by standard techniques, such as the formation of diastereomeric pairs by salt formation with an optically active acid, such as (−)-di-p-toluoyl-D-tartaric acid and/or (+)-di-p-toluoyl-L-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known from the art.

For use in medicine, the salts of the compounds of this invention refer to non-toxic “pharmaceutically acceptable salts.” Other salts may, however, be useful in the preparation of compounds according to this invention or of their pharmaceutically acceptable salts. Suitable pharmaceutically acceptable salts of the compounds include acid addition salts which may, for example, be formed by mixing a solution of the compound with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compounds of the invention carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts. Thus, representative pharmaceutically acceptable salts include the following:

acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate.

Representative acids and bases which may be used in the preparation of pharmaceutically acceptable salts include the following:

acids including acetic acid, 2,2-dichloroactic acid, acylated amino acids, adipic acid, alginic acid, ascorbic acid, L-aspartic acid, benzenesulfonic acid, benzoic acid, 4-acetamidobenzoic acid, (+)-camphoric acid, camphorsulfonic acid, (+)-(1S)-camphor-10-sulfonic acid, capric acid, caproic acid, caprylic acid, cinnamic acid, citric acid, cyclamic acid, dodecylsulfuric acid, ethane-1,2-disulfonic acid, ethanesulfonic acid, 2-hydrocy-ethanesulfonic acid, formic acid, fumaric acid, galactaric acid, gentisic acid, glucoheptonic acid, D-gluconic acid, D-glucoronic acid, L-glutamic acid, α-oxo-glutaric acid, glycolic acid, hipuric acid, hydrobromic acid, hydrochloric acid, (+)-L-lactic acid, (±)-DL-lactic acid, lactobionic acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinc acid, nitric acid, oleic acid, orotic acid, oxalic acid, palmitric acid, pamoic acid, phosphoric acid, L-pyroglutamic acid, salicylic acid, 4-amino-salicylic acid, sebaic acid, stearic acid, succinic acid, sulfuric acid, tannic acid, (+)-L-tartaric acid, thiocyanic acid, p-toluenesulfonic acid and undecylenic acid; and

bases including ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

The present invention is further directed to a process for the preparation of compounds of formula (I-A), as outlined in more detail in Scheme 1 below.

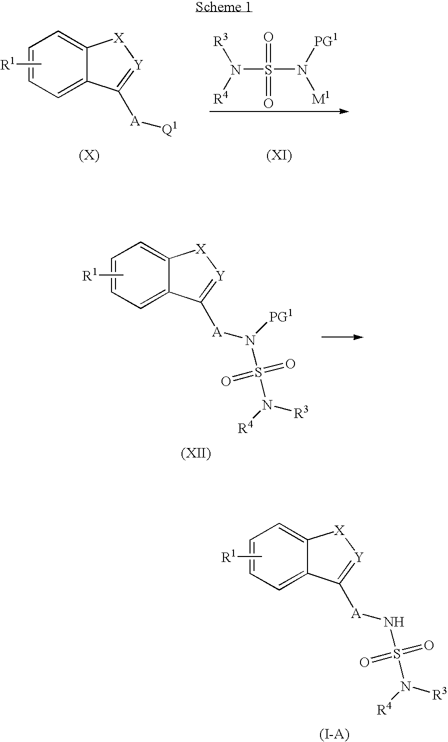

Accordingly, a suitably substituted compound of formula (X), wherein Q¹ is a suitably selected leaving group such as bromo, chloro, iodo, mesylate, tosylate, triflate, and the like, preferably, Q¹ is bromo, chloro, mesylate or tosylate, a known compound or compound prepared by known methods, is reacted with a suitably substituted compound of formula (XI), wherein PG¹ is hydrogen or a suitably selected nitrogen protecting group such as BOC, CBz, and the like, preferably PG¹ is BOC; and wherein M¹ is hydrogen, a known compound or compound prepared by known methods, in the presence of a base such as an inorganic base such as K₂CO₃, Na, Cs₂CO₃, and the like, preferably K₂CO₃ or a tertiary amine base such as NMM, TEA, DIPEA, pyridine, and the like; wherein the base is preferably present in an amount in the range of from about 1.0 to about 5.0 molar equivalents, preferably in the range of from about 4.0 and 5.0 molar equivalents; in an organic solvent, such as toluene, acetone, DMF, and the like; preferably in a polar aprotic solvent, such as acetone, DMF, 2-butanol, and the like, preferably DMF; provided that the compound of formula (X) and the compound of formula (XI) are soluble in the selected organic solvent; to yield the corresponding compound of formula (XII).

Alternatively, a suitably substituted compound of formula (X), wherein Q¹ is a suitably selected leaving group such as bromo, chloro, iodo, mesylate, tosylate, triflate, and the like, preferably, Q¹ is bromo, chloro, mesylate or tosylate, a known compound or compound prepared by known methods, is reacted with a suitably substituted compound of formula (XI), wherein PG¹ is a suitably selected nitrogen protecting group such as BOC, CBz, and the like, preferably PG¹ is BOC; preferably BOC; and wherein M¹ is a metal cation such as sodium cation (Na⁺), potassium cation (K⁺), and the like or is a tertiary ammonium cation such as N-methylmorpholinium, trialkylammonium, (such as triethylammonium) and the like, preferably N-methylmorpholinium; in an organic solvent, such as toluene, acetone, DMF, and the like; preferably in a polar aprotic solvent, such as acetone, DMF, 2-butanol, and the like, preferably DMF; provided that the compound of formula (X) and the compound of formula (XI) are soluble in the selected organic solvent; to yield the corresponding compound of formula (XII).

The compound of formula (XII) is de-protected according to known methods, to yield the corresponding compound of formula (I-A). For example, wherein the compound of formula (XII), PG¹ is BOC, the compound of formula (XII) is de-protected by reacting with a suitably selected acid, such as HCl (for example aqueous HCl), TFA, and the like, in an organic solvent, such as methanol, ethanol, IPA, and the like, to yield the corresponding compound of formula (I-A).

Preferably, the compound of formula (I-A) is isolated according to known methods, for example by extraction with a suitably selected organic solvent such as ethyl acetate, and the like, followed by evaporation of the solvent. Alternatively, the compound of formula (I-A) is further extracted with a solution of NaOH, followed by acidification of the resulting mixture (preferably to a pH in the range of from about 5 to about 7), to yield a precipitate of the compound of formula (I-A). Preferably, the compound of formula (I-A) is purified according to known methods, for example by recrystallization from a suitably selected organic solvent or mixture thereof, such as toluene, IPA, a mixture of MTBE and water, and the like.

In an embodiment, the present invention is directed to a process for the preparation of a compound of formula (I-S), as outlined in more detail in Scheme 2, below.

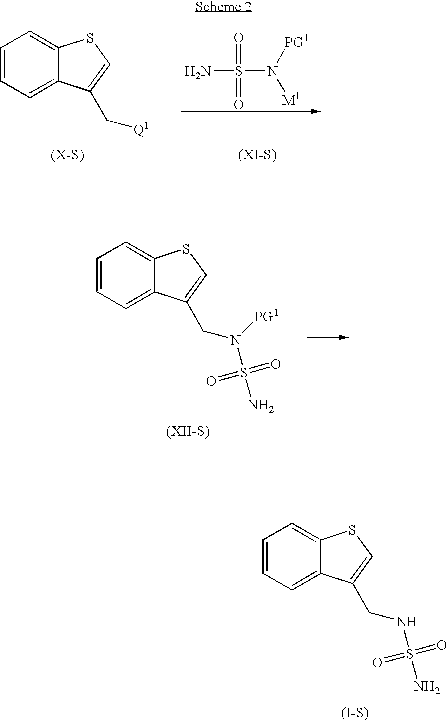

Accordingly, a suitably substituted compound of formula (X-S), wherein Q¹ is a suitably selected leaving group such as bromo, chloro, iodo, mesylate, tosylate, triflate, and the like, preferably, Q¹ is bromo, chloro, mesylate or tosylate, a known compound or compound prepared by known methods, is reacted with a suitably substituted compound of formula (XI-S), wherein PG¹ is hydrogen or a suitably selected nitrogen protecting group such as BOC, CBz, and the like, preferably PG¹ is BOC; and wherein M¹ is hydrogen, a known compound or compound prepared by known methods, in the presence of a base such as an inorganic base such as K₂CO₃, Na, Cs₂CO₃, and the like, preferably K₂CO₃ or a tertiary amine base such as NMM, TEA, DIPEA, pyridine, and the like; wherein the base is preferably present in an amount in the range of from about 1.0 to about 5.0 molar equivalents, preferably in the range of from about 4.0 and 5.0 molar equivalents; in an organic solvent, such as toluene, acetone, DMF, and the like; preferably in a polar aprotic solvent, such as acetone, DMF, 2-butanol, and the like, preferably DMF; provided that the compound of formula (X-S) and the compound of formula (XI-S) are soluble in the selected organic solvent; to yield the corresponding compound of formula (XII-S).

Alternatively, a suitably substituted compound of formula (X-S), wherein Q¹ is a suitably selected leaving group such as bromo, chloro, iodo, mesyltae, tosylate, triflate, and the like, preferably, Q¹ is bromo, chloro, mesylate or tosylate, a known compound or compound prepared by known methods, is reacted with a suitably substituted compound of formula (XI-S), wherein PG¹ is a suitably selected nitrogen protecting group such as BOC, CBz, and the like, preferably PG¹ is BOC; preferably BOC; and wherein M¹ is a metal cation such as sodium cation (Na⁺), potassium cation (K⁺), and the like or is a tertiary ammonium cation such as N-methylmorpholinium, trialkylammonium, (such as triethylammonium) and the like, preferably N-methylmorpholinium; in an organic solvent, such as toluene, acetone, DMF, and the like; preferably in a polar aprotic solvent, such as acetone, DMF, 2-butanol, and the like, preferably DMF; provided that the compound of formula (X-S) and the compound of formula (XI-S) are soluble in the selected organic solvent; to yield the corresponding compound of formula (XII-S).

The compound of formula (XII-S) is de-protected according to known methods, to yield the corresponding compound of formula (I-S). For example, wherein the compound of formula (XII-S), PG¹ is BOC, the compound of formula (XII-S) is de-protected by reacting with a suitably selected acid, such as HCl (for example aqueous HCl), TFA, and the like, in an organic solvent, such as methanol, ethanol, IPA, and the like, to yield the corresponding compound of formula (I-S).

Preferably, the compound of formula (I-S) is isolated according to known methods, for example by extraction with a suitably selected organic solvent such as ethyl acetate, and the like, followed by evaporation of the solvent. Alternatively, the compound of formula (I-S) is further extracted with a solution of NaOH, followed by acidification of the resulting mixture (preferably to a pH in the range of from about 5 to about 7), to yield a precipitate of the compound of formula (I-S). Preferably, the compound of formula (I-S) is purified according to known methods, for example by recrystallization from a suitably selected organic solvent or mixture thereof, such as toluene, IPA, a mixture of MTBE and water, and the like.

The present invention is further directed to a process for the preparation of compounds of formula (Il-A), as outlined in Scheme 3, below.

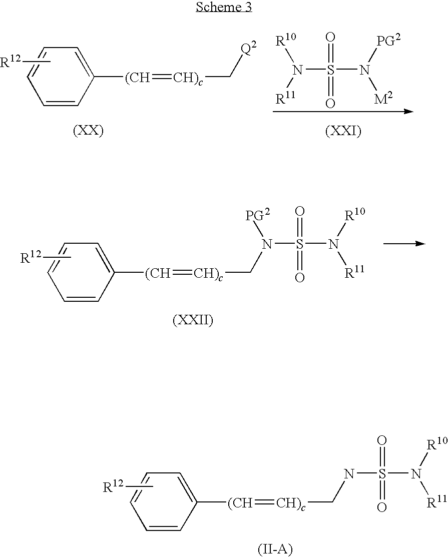

Accordingly, a suitably substituted compound of formula (XX), wherein Q² is a suitably selected leaving group such as bromo, chloro, iodo, mesylate, tosylate, triflate, and the like, preferably, Q² is bromo, chloro, mesylate or tosylate, a known compound or compound prepared by known methods, is reacted with a suitably substituted compound of formula (XXI), wherein PG² is hydrogen or a suitably selected nitrogen protecting group such as BOC, CBz, and the like, preferably PG² is BOC; and wherein M² is hydrogen, a known compound or compound prepared by known methods, in the presence of a base such as an inorganic base such as K₂CO₃, Na, Cs₂CO₃, and the like, preferably K₂CO₃ or a tertiary amine base such as NMM, TEA, DIPEA, pyridine, and the like; wherein the base is preferably present in an amount in the range of from about 1.0 to about 5.0 molar equivalents, preferably in the range of from about 4.0 and 5.0 molar equivalents; in an organic solvent, such as toluene, acetone, DMF, and the like; preferably in a polar aprotic solvent, such as acetone, DMF, 2-butanol, and the like, preferably DMF; provided that the compound of formula (XX) and the compound of formula (XXI) are soluble in the selected organic solvent; to yield the corresponding compound of formula (XXII).

Alternatively, a suitably substituted compound of formula (XX), wherein Q² is a suitably selected leaving group such as bromo, chloro, iodo, mesyltae, tosylate, triflate, and the like, preferably, Q² is bromo, chloro, mesylate or tosylate, a known compound or compound prepared by known methods, is reacted with a suitably substituted compound of formula (XXI), wherein PG² is a suitably selected nitrogen protecting group such as BOC, CBz, and the like, preferably PG² is BOC; preferably BOC; and wherein M² is a metal cation such as sodium cation (Na⁺), potassium cation (K⁺), and the like or is a tertiary ammonium cation such as N-methylmorpholinium, trialkylammonium, (such as triethylammonium) and the like, preferably N-methylmorpholinium; in an organic solvent, such as toluene, acetone, DMF, and the like; preferably in a polar aprotic solvent, such as acetone, DMF, 2-butanol, and the like, preferably DMF; provided that the compound of formula (XX) and the compound of formula (XXI) are soluble in the selected organic solvent; to yield the corresponding compound of formula (XXII).

The compound of formula (XXII) is de-protected according to known methods, to yield the corresponding compound of formula (II-A). For example, wherein the compound of formula (XXII), PG¹ is BOC, the compound of formula (XXII) is de-protected by reacting with a suitably selected acid, such as HCl (for example aqueous HCl), TFA, and the like, in an organic solvent, such as methanol, ethanol, IPA, and the like, to yield the corresponding compound of formula (II-A).

Preferably, the compound of formula (II-A) is isolated according to known methods, for example by extraction with a suitably selected organic solvent such as ethyl acetate, and the like, followed by evaporation of the solvent. Alternatively, the compound of formula (II-A) is further extracted with a solution of NaOH, followed by acidification of the resulting mixture (preferably to a pH in the range of from about 5 to about 7), to yield a precipitate of the compound of formula (II-A). Preferably, the compound of formula (II-A) is purified according to known methods, for example by recrystallization from a suitably selected organic solvent or mixture thereof, such as toluene, IPA, a mixture of MTBE and water, and the like.

The present invention further comprises pharmaceutical compositions containing one or more compounds prepared according to any of the processes described herein with a pharmaceutically acceptable carrier. Pharmaceutical compositions containing one or more of the compounds of the invention described herein as the active ingredient can be prepared by intimately mixing the compound or compounds with a pharmaceutical carrier according to conventional pharmaceutical compounding techniques. The carrier may take a wide variety of forms depending upon the desired route of administration (e.g., oral, parenteral). Thus for liquid oral preparations such as suspensions, elixirs and solutions, suitable carriers and additives include water, glycols, oils, alcohols, flavoring agents, preservatives, stabilizers, coloring agents and the like; for solid oral preparations, such as powders, capsules and tablets, suitable carriers and additives include starches, sugars, diluents, granulating agents, lubricants, binders, disintegrating agents and the like. Solid oral preparations may also be coated with substances such as sugars or be enteric-coated so as to modulate major site of absorption. For parenteral administration, the carrier will usually consist of sterile water and other ingredients may be added to increase solubility or preservation. Injectable suspensions or solutions may also be prepared utilizing aqueous carriers along with appropriate additives.

To prepare the pharmaceutical compositions of this invention, one or more compounds of the present invention as the active ingredient is intimately admixed with a pharmaceutical carrier according to conventional pharmaceutical compounding techniques, which carrier may take a wide variety of forms depending of the form of preparation desired for administration, e.g., oral or parenteral such as intramuscular. In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules, caplets, gelcaps and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed. If desired, tablets may be sugar coated or enteric coated by standard techniques. For parenterals, the carrier will usually comprise sterile water, through other ingredients, for example, for purposes such as aiding solubility or for preservation, may be included. Injectable suspensions may also be prepared, in which case appropriate liquid carriers, suspending agents and the like may be employed. The pharmaceutical compositions herein will contain, per dosage unit, e.g., tablet, capsule, powder, injection, teaspoonful and the like, an amount of the active ingredient necessary to deliver an effective dose as described above. The pharmaceutical compositions herein will contain, per unit dosage unit, e.g., tablet, capsule, powder, injection, suppository, teaspoonful and the like, of from about 0.01-10,000 mg or any range therein, and may be given at a dosage of from about 0.01-500 mg/kg/day, or any range therein, preferably from about 1.0-50 mg/kg/day, or any range therein. The dosages, however, may be varied depending upon the requirement of the patients, the severity of the condition being treated and the compound being employed. The use of either daily administration or post-periodic dosing may be employed.

Preferably these compositions are in unit dosage forms from such as tablets, pills, capsules, powders, granules, sterile parenteral solutions or suspensions, metered aerosol or liquid sprays, drops, ampoules, autoinjector devices or suppositories; for oral parenteral, intranasal, sublingual or rectal administration, or for administration by inhalation or insufflation. Alternatively, the composition may be presented in a form suitable for once-weekly or once-monthly administration; for example, an insoluble salt of the active compound, such as the decanoate salt, may be adapted to provide a depot preparation for intramuscular injection. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of material can be used for such enteric layers or coatings, such materials including a number of polymeric acids with such materials as shellac, cetyl alcohol and cellulose acetate.

The liquid forms in which the novel compositions of the present invention may be incorporated for administration orally or by injection include, aqueous solutions, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone or gelatin.

The method of treating epilepsy and related disorders described in the present invention may also be carried out using a pharmaceutical composition comprising any of the compounds as defined herein and a pharmaceutically acceptable carrier. The pharmaceutical composition may contain between about 0.01 mg and 1000 mg of the compound, or any range therein; preferably about 10 to 500 mg of the compound, and may be constituted into any form suitable for the mode of administration selected. Carriers include necessary and inert pharmaceutical excipients, including, but not limited to, binders, suspending agents, lubricants, flavorants, sweeteners, preservatives, dyes, and coatings. Compositions suitable for oral administration include solid forms, such as pills, tablets, caplets, capsules (each including immediate release, timed release and sustained release formulations), granules, and powders, and liquid forms, such as solutions, syrups, elixers, emulsions, and suspensions. Forms useful for parenteral administration include sterile solutions, emulsions and suspensions.

Advantageously, compounds of the present invention may be administered in a single daily dose, or the total daily dosage may be administered in divided doses of two, three or four times daily. Furthermore, compounds for the present invention can be administered in intranasal form via topical use of suitable intranasal vehicles, or via transdermal skin patches well known to those of ordinary skill in that art. To be administered in the form of a transdermal delivery system, the dosage administration will, of course, be continuous rather than intermittent throughout the dosage regimen.

For instance, for oral administration in the form of a tablet or capsule, the active drug component can be combined with an oral, non-toxic pharmaceutically acceptable inert carrier such as ethanol, glycerol, water and the like. Moreover, when desired or necessary, suitable binders; lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The liquid forms in suitably flavored suspending or dispersing agents such as the synthetic and natural gums, for example, tragacanth, acacia, methyl-cellulose and the like. For parenteral administration, sterile suspensions and solutions are desired. Isotonic preparations which generally contain suitable preservatives are employed when intravenous administration is desired.

To prepare a pharmaceutical composition of the present invention, a compound of formula (I) as the active ingredient is intimately admixed with a pharmaceutical carrier according to conventional pharmaceutical compounding techniques, which carrier may take a wide variety of forms depending of the form of preparation desired for administration (e.g. oral or parenteral). Suitable pharmaceutically acceptable carriers are well known in the art. Descriptions of some of these pharmaceutically acceptable carriers may be found in The Handbook of Pharmaceutical Excipients, published by the American Pharmaceutical Association and the Pharmaceutical Society of Great Britain.

Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc.

Compounds of this invention may be administered in any of the foregoing compositions and according to dosage regimens established in the art whenever treatment of epilepsy and related disorders is required.

The daily dosage of a product prepared according to any of the processes described herein may be varied over a wide range from 0.01 to 10,000 mg per adult human per day, or any range therein. For oral administration, the compositions are preferably provided in the form of tablets containing, 0.01, 0.05, 0.1, 0.5, 1.0, 2.5, 5.0, 10.0, 15.0, 25.0, 50.0, 100, 150, 200, 250 and 500 milligrams of the active ingredient for the symptomatic adjustment of the dosage to the patient to be treated. An effective amount of the drug is ordinarily supplied at a dosage level of from about 0.01 mg/kg to about 500 mg/kg of body weight per day, or any range therein. Preferably, the range is from about 0.5 to about 250 mg/kg of body weight per day, or any range therein. More preferably, from about 1.0 to about 100 mg/kg of body weight per day, or any range therein. More preferably, from about 1.0 to about 50 mg/kg of body weight per day, or any range therein. The compounds may be administered on a regimen of 1 to 4 times per day.

Optimal dosages to be administered may be readily determined by those skilled in the art, and will vary with the particular compound used, the mode of administration, the strength of the preparation, the mode of administration, and the advancement of the disease condition. In addition, factors associated with the particular patient being treated, including patient age, weight, diet and time of administration, will result in the need to adjust dosages.

One skilled in the art will recognize that, both in vivo and in vitro trials using suitable, known and generally accepted cell and/or animal models are predictive of the ability of a test compound to treat or prevent a given disorder.

One skilled in the art will further recognize that human clinical trails including first-in-human, dose ranging and efficacy trials, in healthy patients and/or those suffering from a given disorder, may be completed according to methods well known in the clinical and medical arts.

The following Examples are set forth to aid in the understanding of the invention, and are not intended and should not be construed to limit in any way the invention set forth in the claims which follow thereafter.

In the Examples which follow, some synthesis products are listed as having been isolated as a residue. It will be understood by one of ordinary skill in the art that the term “residue” does not limit the physical state in which the product was isolated and may include, for example, a solid, an oil, a foam, a gum, a syrup, and the like. All melting points were determined using a TA-Q100 Differential Scanning Calorimetry (DSC) instrument.

### EXAMPLE 1

### tert-Butyl sulfamoylcarbamate (Boc-sulfamide)

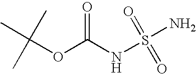

tert-Butyl sulfamoylcarbamate (Boc-sulfamide) was prepared using the procedure of Masui, et al, [Masui, T; Kabaki, M.; Watanabe, H.; Kobayashi, T.; Masui, Y., Org. Process Res. Dev. 2004, 8, 408-410].

### EXAMPLE 2

### tert-Butyl sulfamoylcarbamate sodium salt

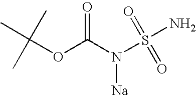

tert-Butyl sulfamoylcarbamate (6.0 g 30.58 mmol) was placed in a 100 mL round-bottomed flask together with methanol (50 mL) and sodium hydroxide (2.45 g; 30.63 mmol). After stirring for a few minutes, the solvent was evaporated under reduced pressure to yield a white solid. The solid was dissolved in methanol (50 mL) with heating. The resulting mixture was hot-filtered through Celite® to remove some fine insoluble solid, to yield a clear solution. The solvent was evaporated and the remaining solid product was recrystallized from EtOAc/MeOH. The resulting crystalline solid was collected by filtration and air dried to yield the title compound.

mp: 224° C.

¹H NMR (d₆-DMSO): δ5.19 (s, 2H), 1.31 (s, 9H)

### EXAMPLE 3

### tert-Butyl sulfamoylcarbamate N-methyl morpholine salt

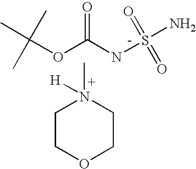

tert-Butyl sulfamoylcarbamate (6 g, 30.58 mmol) was placed in a 100 mL round bottomed flask together with methanol (50 mL) and N-methylmorpholine (6.19 g, 6.75 mL, 61.15 mmol). The resulting mixture was stirred at room temperature for about 10-15 minutes. Most of the solvent was evaporated under reduced pressure at 30° C. to about 10-15 mL final volume. The resulting solution was diluted with ethyl acetate (˜40 mL) and most of the solvent was evaporated to about 15 mL final volume and then allowed to stand at room temperature. The product started to precipitate as a crystalline white solid. Heptane was added slowly to insure maximum precipitation. The solid was collected by filtration, rinsed with heptane containing 2-3% EtOAc and then air dried to yield the title compound.

mp: 100° C.

¹H NMR (d₆-DMSO): δ10.78 (bs, 1H), 7.23 (s, 2H), 3.56 (t, J=4.6 Hz, 4H), 2.33-2.26 (m, 4H), 2.16 (s, 3H), 1.43 (s, 9H)

Elemental analysis, calculated for: C₁₀H₂₃N₃O₅S: C, 40.39; H, 7.80; N, 14.13; S, 10.78. Found: C, 39.88, H, 7.97, N, 14.08, S, 10.85.

### EXAMPLE 4

### tert-Butyl benzyl(sulfamoyl)carbamate

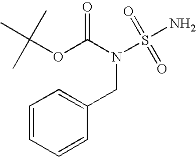

Benzyl bromide (3.42 g, 20.0 mmol) was placed in a 125 mL Erlenmeyer flask together with N,N-dimethylformamide (50 mL), Boc-sulfamide (4.32 g, 22.0 mmol), and potassium carbonate (11.06 g, 80 mmol). The resulting mixture was stirred at room temperature for 1 h. The progress of reaction was monitored by TLC analysis on silica gel plates using EtOAc/Heptane (1:1) as eluent. The resulting mixture was filtered to remove the solid carbonate and the filtrate was poured into water (300 mL), acidified with AcOH, then allowed to stand for 10 min. The product precipitated as a white crystalline solid. The solid was collected by filtration, washed with water and air-dried to yield the title compound.

mp: 127.3° C.

¹H NMR (d₆-DMSO): δ 7.60 (s, 2H), 7.38-7.22 (m, 5H), 4.76 (s, 2H), 1.36 (s, 9H)

Elemental Analysis for C₁₂H₁₈N₂O₄S: Calculated: C, 50.33; H, 6.34; N, 9.78; S, 11.20. Found: C, 50.09; H, 6.44; N, 9.72; S, 10.88.

### EXAMPLE 5

### Benzylsulfamide

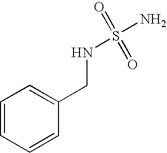

N-Benzyl-N-Boc-sulfamide (5.0 g, 17.46 mmol) was mixed with conc. aq. HCl (25 mL) in a 125 mL flask equipped with a nitrogen inlet and a magnetic stir bar. The resulting mixture was stirred overnight at room temperature. The progress of reaction was monitored by TLC analysis on silica gel plates using EtOAc/Heptane (1:1) as eluent. The resulting suspension was diluted with water (40 mL) and the product was extracted with EtOAc (2×50 mL). The EtOAc extract was washed with aq. Sat. NaHCO₃ solution and dried (Na₂SO₄). Evaporation of the solvent yielded the title compound as a white solid. The solid was further purified by recrystallization from toluene.

mp 109.7° C. [Lit. (Aeberli, P; Gogerty, J; Houlihan, W. J., J. Med. Chem, 10 (4), 1967, 636-642), mp, 102-4° C (EtOH/H₂O)]

¹H NMR (d₆-DMSO): δ 7.38-7.21 (m, 5H), 7.05 (t, J=6.6 Hz, 1H), 6.63 (s, 2H), 4.07 (d, J=6.6 Hz, 2H)

Elemental Analysis, calculated for C₇H₁₀N₂O₂S: C, 45.15; H, 5.41; N, 15.04; O, 17.18; S, 17.22. Found: C, 45.68, H, 5.19, N, 14.84, S, 16.73.

### EXAMPLE 6

### tert-Butyl cinnamyl(sulfamoyl)carbamate

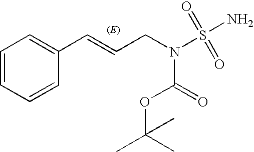

Cinnamyl bromide (2.96 g, 15.0 mmol) was placed in a 100 mL round-bottomed flask together with N,N-dimethylformamide (30 mL), Boc-sulfamide (3.24g, 16.5 mmol), and potassium carbonate (8.3 g, 60 mmol). The resulting mixture was stirred at room temperature for 1 h. The progress of reaction was monitored by TLC analysis on silica gel plates using EtOAc/Heptane (1:1) as eluent. The resulting mixture was filtered to remove the solid carbonate and the filtrate was poured into ice-water (200 mL) and then allowed to stand for 10 min. The product precipitated as a white solid. The solid was collected by filtration, washed with water and air-dried to yield the title compound. The solid was further purified by recrystallization from toluene.

mp: 117.6° C.

¹H NMR (d₆-DMSO): δ7.54 (s, 2H), 7.43 (d, J=7.2 Hz, 2H), 7.34 (t, J=7.2 Hz, 2H), 7.25 (t, J=7.2 Hz, 1H), 6.52 (d, J=15.8 Hz, 1H), 6.29 (dt, J₁=5.9, J₂=15.8 Hz, 1H), 4.32 (d, J=5.9 Hz, 2H), 1.46 (s, 9H)

¹³C NMR (d₆-DMSO): δ151.3 (C), 136.3 (C), 131.6 (CH), 128.6 (CH), 127.6 (CH), 126.3 (CH), 125.3 (CH), 82.4 (C), 48.9 (CH₂), 27.7 (CH₃)

Elemental analysis, calculated for C₁₄H₂₀N₂O₄S: C, 53.83; H, 6.45; N, 8.97; S, 10.26. Found: C, 54.06, H, 6.50, N, 8.82, S, 10.10.

### EXAMPLE 7

### Cinnamylsulfamide

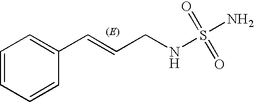

tert-Butyl cinnamyl(sulfamoyl)carbamate (2.5 g, 8 mmol) was dissolved in 4N HCl in dioxane (15 mL) and stirred at room temperature for 1 h. The resulting solution was poured onto ice cold water (70 mL) and neutralized by adding solid NaHCO₃ in small portions. The precipitated solid was collected by filtration, washed with cold water and air-dried. The solid was further purified by recrystallization from toluene to yield the title compound.

mp: 117.9° C.

¹H NMR (d₆-DMSO): δ7.41 (d, J=7.3 Hz, 2H), 7.34 (t, J=7.7 Hz, 2H), 7.24 (t, J=6.8 Hz, 1H), 6.64 (bs, 2H), 6.56 (d, J=16.2 Hz, 2H), 6.30 (dt, J₁=6.0, J₂=16.2 Hz, 1H), 3.68 (d, J=6.0 Hz, 2H)

¹³C NMR (d₆-DMSO): δ136.6 (C), 130.6 (CH), 128.6 (CH), 127.4 (CH), 126.8 (CH), 126.1 (CH), 44.6 (CH₂).

### EXAMPLE 8

### 3-(Bromomethyl)benzo[b]thiophene

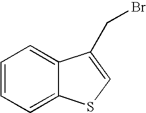

Benzo[b]thiophen-3-ylmethanol (16.4 g, 100 mmol) was placed in a 500 mL round bottomed flask together with toluene (150 mL), and concentrated (48%) aq. hydrogen bromide (100 mL, 890 mmol) and stirred at room temperature for 1 h. The progress of reaction was monitored by TLC analysis for complete conversion. The organic layer was separated, washed with water and aq. NaHCO₃ and dried (MgSO₄). The solvent was stirred with a small amount of silica gel to remove some light brown color, filtered and evaporated under reduced pressure at 35-40° C. to about a final volume of 30 mL and then allowed to stand at room temperature. The title compound crystallized out as a light yellow solid. Heptane (100 mL) was added slowly to ensure complete crystallization, the solid was collected by filtration and rinsed with heptane then air-dried to yield the title compound.

¹H NMR (d₆-DMSO): δ 7.99 (dd, J1=7.6, J2=26.2 Hz, 2H), 7.97(s, 1H), 7-54-7.39 (m, 2H), 5.03 (s, 2H)

### EXAMPLE 9

### N-(benzo[b]thien-3-ylmethyl)sulfamide

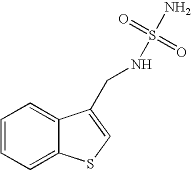

Step A: tert-Butyl benzo[b]thiophen-3-ylmethyl(sulfamoyl)carbamate

3-(bromomethyl)benzo[b]thiophene (4.54 g, 20.0 mmol) was placed in a 100 mL round bottomed flask equipped with a nitrogen inlet and a magnetic stir bar. N,N-Dimethylformamide (100 mL), Boc-sulfamide (4.32 g, 22.0 mmol), and potassium carbonate (11.06 g, 80.0 mmol) were added and the resulting mixture was stirred at room temperature for 4 h. The progress of reaction was monitored by TLC analysis on silica gel plates using EtOAc/Heptane (1:1) as the eluent. The resulting mixture was quenched with cold water and the product was extracted with EtOAc. The EtOAc extract was washed with aq. sat. sodium bicarbonate solution then dried with MgSO₄. Evaporation of the solvent under reduced pressure yielded tert-butyl benzo[b]thiophen-3-ylmethyl(sulfamoyl)carbamate as a white solid.

mp 102.6° C.

¹H NMR (d₆-DMSO): δ8.03-7.96 (m, 1H), 7.96-7.89 (m, 1H), 7.68 (s, 2H), 7.47 (s,1H), 7.46-7.35 (m, 2H), 5.01 (s, 2H), 1.36 (s, 9H)

¹³C NMR (d₆-DMSO): δ151.4 (C), 139.7 (C), 137.2 (C), 133.0 (C), 124.5 (CH), 124.1 (CH), 123.4 (CH), 122.9 (CH), 121.7 (CH), 82.8 (C), 45.0 (CH₂), 27.6 (CH₃). Elemental analysis, calculated for C₁₄H₁₈N₂O₄S₂, C, 49.10; H, 5.30; N, 8.18; S, 18.73. Found: C, 49.51, H, 5.22, N, 8.05, S, 19.01.

### Step B: N-(benzo[b]thien-3-ylmethyl)sulfamide

The solid from step A (6.4 g, 187 mmol) was dissolved in MeOH and treated with conc. HCl. The resulting mixture was stirred for 2 h at room temperature. The solvent was evaporated under reduced pressure and the product was dissolved in aq 1N NaOH and extracted with ethyl acetate. The aqueous layer was cooled and acidified to pH about 6-8 with 1N HCl. The resulting solid was collected by filtration, washed with water and air-dried to yield the title compound.

mp 101.4° C.

### EXAMPLE 10

### N-(benzo[b]thien-3-ylmethyl)sulfamide

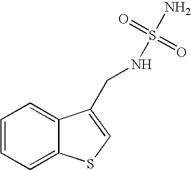

A 500 mL round bottomed flask was equipped with a stir bar, nitrogen outlet, heating mantle and temperature control unit was charged with:3-Chloromethyl-benzo[b]thiophene (20.0 g, 109.5 mmol), sulfamide (32.54 g, 328.5 mmol), N,N-dimethylformamide (125 mL) followed by addition of potassium carbonate (22.7 g, 164.2 mmol) and tetra(n-butyl)ammonium iodide (1.01 g, 2.74 mmol). The resulting mixture was then warmed to 40° C. and stirred overnight. The reaction was treated with another equivalent of sulfamide (11 g) and K₂CO₃ (7 g) then warmed to 50° C. and stirred overnight to drive the reaction to completion. The resulting mixture was filtered then treated with water (100 mL) and 1N HCl (10 mL). The desired product was extracted with MTBE (2×100 mL) and the organic layer washed with water (50 mL). The aqueous layer was then treated with NaCl (15 g) and extracted with EtOAc (2×100 mL). The MTBE and EtOAc layers were combined, dried (Na₂SO₄), filtered and concentrated to yield a thick oil (29 g). The oil was dissolved in 1N NaOH (20 mL) and the aqueous solution was extracted with MTBE to remove any non-acidic impurities. The desired product was recovered by acidification of aqueous layer and extraction with MTBE. The MTBE was dried and concentrated to yield a solid (20 g). The solid was heated in water (1500 mL), the resulting solution was hot filtered and the filtrate was cooled slowly to room temperature. The product precipitated as a white solid, after filtration and drying under vacuum, the title compound was isolated as a solid.

### EXAMPLE 11

As a specific embodiment of an oral composition, 100 mg of the compound prepared as in Example 9 or Example 10 is formulated with sufficient finely divided lactose to provide a total amount of 580 to 590 mg to fill a size O hard gel capsule.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

We claim:

1. A process for the preparation of compounds of formula (I-A) 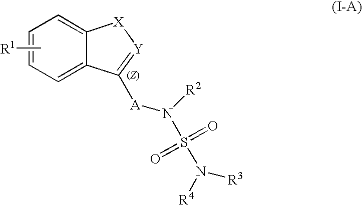
wherein
R¹ is selected from the group consisting of hydrogen, halogen, hydroxy, methoxy, trifluoromethyl, nitro and cyano;
X—Y is selected from the group consisting of —S—CH—, —S—C(CH₃)—, —O—CH—, —O—C(CH₃)—, —N(CH₃)—CH— and —CH═CH—CH—;
A is selected from the group consisting of —CH₂— and —CH(CH₃)—;
R² is hydrogen;
R³ and R⁴ are each independently selected from the group consisting of hydrogen and C₁₋₄alkyl;
alternatively, R³ and R⁴ are taken together with the nitrogen atom to which they are bound to form a 5 to 7 membered, saturated, partially unsaturated or aromatic ring structure, optionally containing one to three additional heteroatoms independently selected from the group consisting of O, N and S;
or a pharmaceutically acceptable salt thereof; comprising 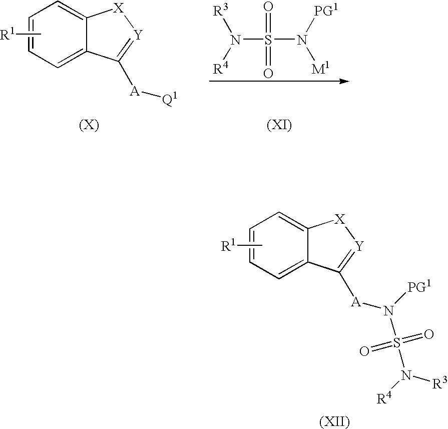
reacting a compound of formula (X), wherein Q¹ is a leaving group with a compound of formula (XI), wherein PG¹ is hydrogen or a nitrogen protecting group, and wherein M¹ is hydrogen; in the presence of a base; in an organic solvent; to yield the corresponding compound of formula (XII); 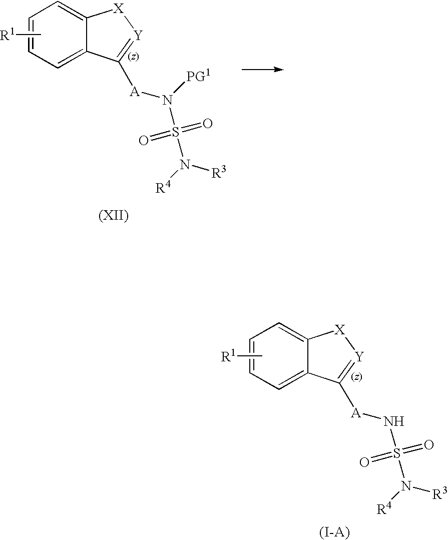
de-protecting the compound of formula (XII), to yield the corresponding compound of formula (I-A).

2. A process as in claim 1, wherein Q¹ is selected from the group consisting of chloro, bromo, mesylate and tosylate; PG¹ is Boc; and M¹ is hydrogen.

3. A process as in claim 1, wherein the base is an inorganic base.

4. A process as in claim 1, wherein the base K₂CO₃ and is present in an amount in the range of from about 1.0 to about 5.0 molar equivalents.

5. A process as in claim 1, wherein the organic solvent is DMF.

6. A process for the preparation of compounds of formula (I-A) 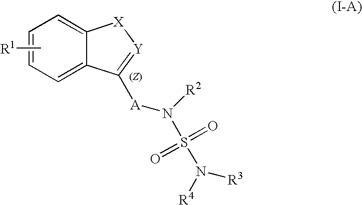
wherein
R¹ is selected from the group consisting of hydrogen, halogen, hydroxy, methoxy, trifluoromethyl, nitro and cyano;
X—Y is selected from the group consisting of —S—CH—, —S—C(CH₃)—, —O—CH—, —O—C(CH₃)—, —N(CH₃)—CH— and —CH═CH—CH—;
A is selected from the group consisting of —CH₂— and —CH(CH₃)—;
R² is hydrogen;
R³ and R⁴ are each independently selected from the group consisting of hydrogen and C₁₋₄alkyl;
alternatively, R³ and R⁴ are taken together with the nitrogen atom to which they are bound to form a 5 to 7 membered, saturated, partially unsaturated or aromatic ring structure, optionally containing one to three additional heteroatoms independently selected from the group consisting of O, N and S;
or a pharmaceutically acceptable salt thereof; comprising 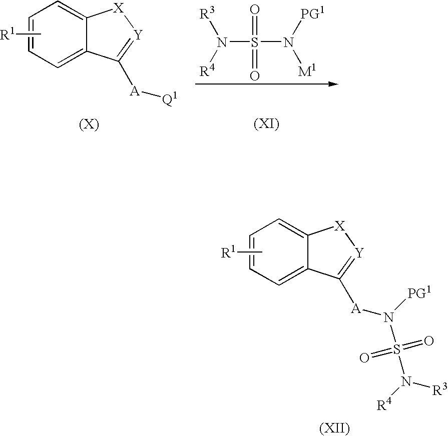
or reacting a compound of formula (X), wherein Q¹ is a leaving group with a compound of formula (XI), wherein PG¹ is a nitrogen protecting group, and wherein M¹ is a metal cation or a tertiary ammonium ion; in an organic solvent; to yield the corresponding compound of formula (XII); 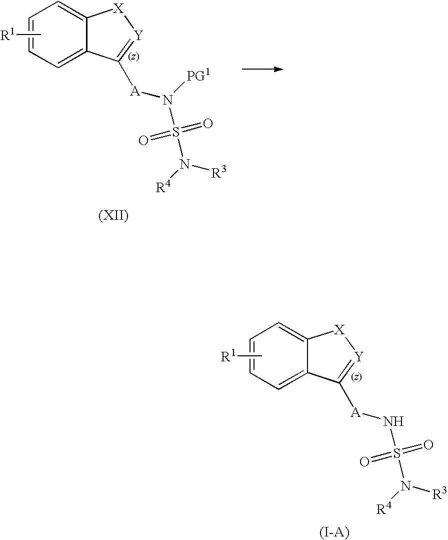
de-protecting the compound of formula (XII), to yield the corresponding compound of formula (I-A).

7. A process as in claim 6, wherein Q1 is selected from the group consisting of chloro, bromo, mesylate and tosylate; PG¹ is BOC; and M¹ is N-methylmorpholinium.

8. A process as in claim 6, wherein the organic solvent is DMF.

9. A process for the preparation of a compound of formula (I-S) 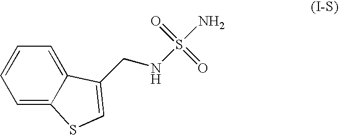
or a pharmaceutically acceptable salt thereof; comprising 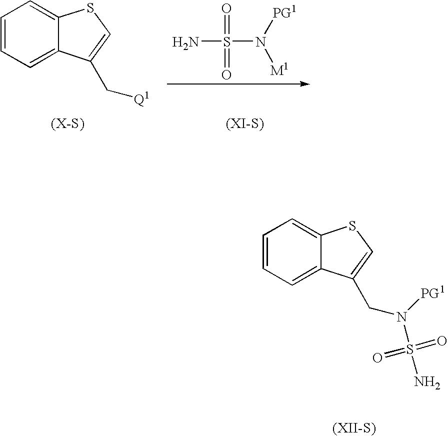
reacting a compound of formula (X-S), wherein Q¹ is a leaving group with a compound of formula (XI-S), wherein PG¹ is hydrogen or a nitrogen protecting group, and wherein M¹ is hydrogen; in the presence of a base; in an organic solvent; to yield the corresponding compound of formula (XII-S); 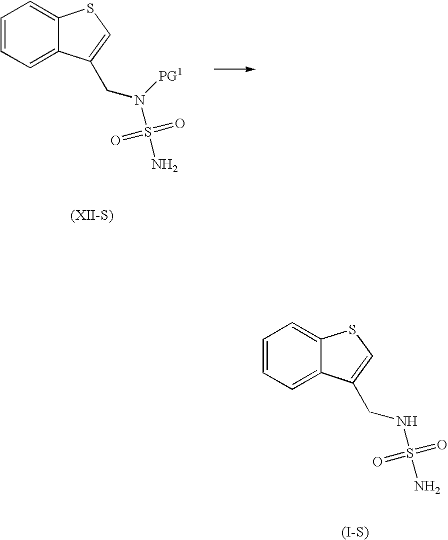
de-protecting the compound of formula (XII-S), to yield the corresponding compound of formula (I-S).

10. A process as in claim 9, wherein Q¹ is Br.

11. A process as in claim 9, wherein PG¹ is BOC.

12. A process as in claim 9, wherein M¹ is hydrogen.

13. A process as in claim 9, wherein the base is an inorganic base.

14. A process as in claim 13, wherein the inorganic base is K₂CO₃.

15. A process as in claim 9, and wherein the base is present in an amount in the range of from about 1.0 to about 5.0 molar equivalents.

16. A process as in claim 15, and wherein the base is present in an amount in the range of from about 4.0 to about 5.0 molar equivalents.

17. A process as in claim 9, wherein the organic solvent is DMF.

18. A process for the preparation of a compound of formula (I-S) 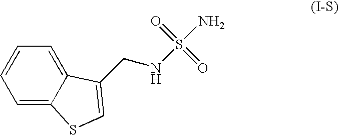
or a pharmaceutically acceptable salt thereof; comprising 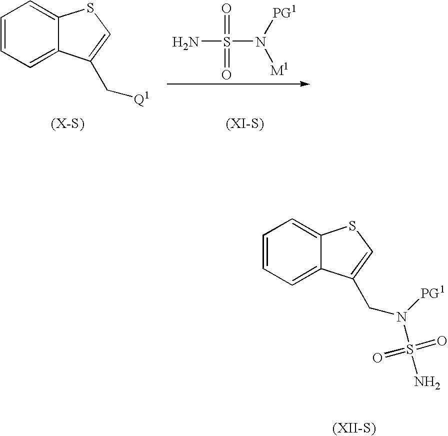
or reacting a compound of formula (X-S), wherein Q¹ is a leaving group with a compound of formula (XI-S), wherein PG¹ is a nitrogen protecting group, and wherein M¹ is a metal cation or a tertiary ammonium ion; in an organic solvent; to yield the corresponding compound of formula (XII-S); 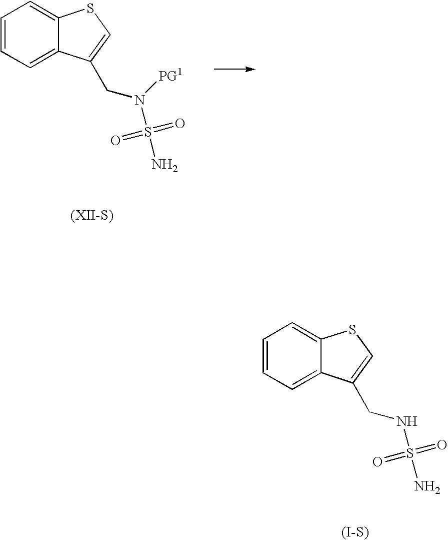
de-protecting the compound of formula (XII-S), to yield the corresponding compound of formula (I-S).

19. A process as in claim 18, wherein Q¹ is Br.

20. A process as in claim 18, wherein PG¹ is BOC.

21. A process as in claim 18, wherein M¹ is a tertiary ammonium cation.

22. A process as in claim 18, wherein M¹ is N-methylmorpholinium.

23. A process as in claim 18, wherein the organic solvent is DMF.

24. A process for the preparation of a compound of formula (II-A) 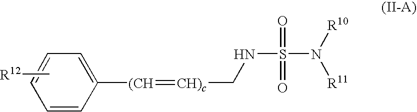
wherein
R¹² is selected from the group consisting of hydrogen, halogen, hydroxy, methoxy, trifluoromethyl, nitro and cyano;
c is an integer from 0 to 2;
R¹⁰ and R¹¹ are each independently selected from the group consisting of hydrogen and C₁₋₄alkyl;
alternatively, R¹⁰ and R¹¹ are taken together with the nitrogen atom to which they are bound to form a 5 to 7 membered, saturated, partially unsaturated or aromatic ring structure, optionally containing one to three additional heteroatoms independently selected from the group consisting of O, N and S;
or a pharmaceutically acceptable salt thereof; comprising 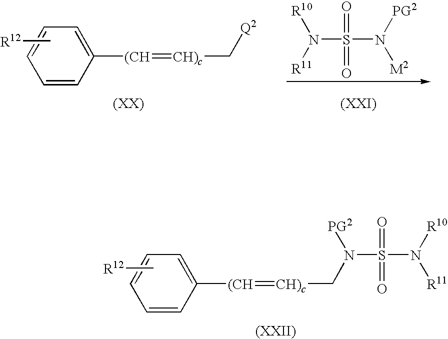
reacting a compound of formula (XX), wherein Q² is a leaving group with a compound of formula (XXI), wherein PG² is hydrogen or a nitrogen protecting group, and wherein M² is hydrogen; in the presence of a base; in an organic solvent; to yield the corresponding compound of formula (XXII); 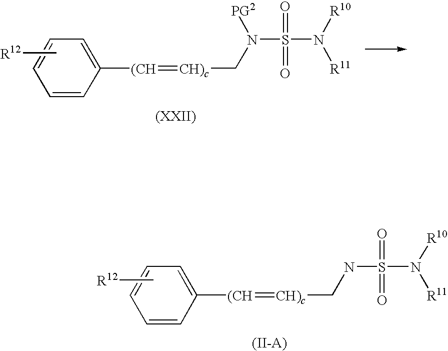
de-protecting the compound of formula (XXII), to yield the corresponding compound of formula (II-A).

25. A process as in claim 24, wherein R¹², R¹⁰ and R¹¹ are each hydrogen.

26 A process for the preparation of a compound of formula (II-A) 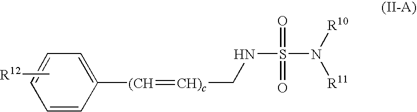
wherein
R¹² is selected from the group consisting of hydrogen, halogen, hydroxy, methoxy, trifluoromethyl, nitro and cyano;
c is an integer from 0 to 2;
R¹⁰ and R¹¹ are each independently selected from the group consisting of hydrogen and C₁₋₄alkyl;
alternatively, R¹⁰ and R¹¹ are taken together with the nitrogen atom to which they are bound to form a 5 to 7 membered, saturated, partially unsaturated or aromatic ring structure, optionally containing one to three additional heteroatoms independently selected from the group consisting of O, N and S;
or a pharmaceutically acceptable salt thereof; comprising 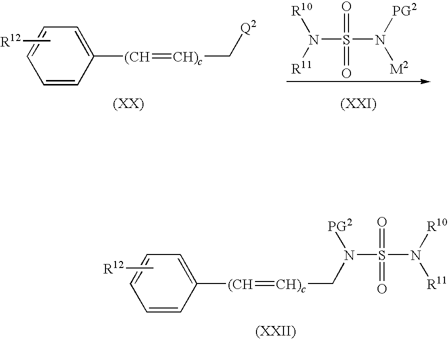
or reacting a compound of formula (XX), wherein Q² is a leaving group with a compound of formula (XXI), wherein PG² is a nitrogen protecting group, and wherein M¹ is a metal cation or a tertiary ammonium ion; in an organic solvent; to yield the corresponding compound of formula (XXII); 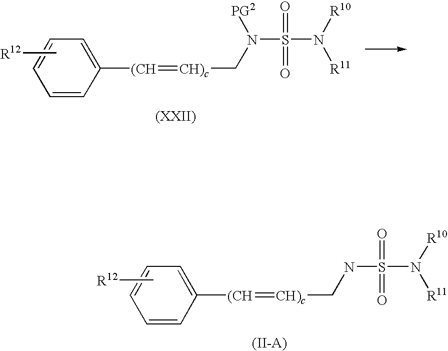
de-protecting the compound of formula (XXII), to yield the corresponding compound of formula (II-A).

27. A process as in claim 26, wherein R¹², R¹⁰ and R¹¹ are each hydrogen.

28. A compound of formula (II-A) 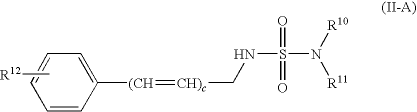
wherein
R¹² is selected from the group consisting of hydrogen, halogen, hydroxy, methoxy, trifluoromethyl, nitro and cyano;
c is an integer from 0 to 2;
R¹⁰ and R¹¹ are each independently selected from the group consisting of hydrogen and C₁₋₄alkyl;
alternatively, R¹⁰ and R¹¹ are taken together with the nitrogen atom to which they are bound to form a 5 to 7 membered, saturated, partially unsaturated or aromatic ring structure, optionally containing one to three additional heteroatoms independently selected from the group consisting of O, N and S;
provided that when c is 0; then R¹² is other than hydrogen;
or a pharmaceutically acceptable salt thereof.

29. A compound as in claim 28, wherein R¹² is hydrogen.

30. A compound as in claim 28, wherein R¹² is hydrogen, R¹⁰ is hydrogen, R¹¹ is hydrogen and c is an integer from 1 to 2.

31. A compound of formula (XII) 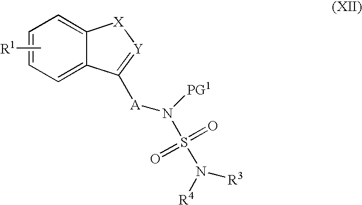
wherein
PG¹ is hydrogen or a nitrogen protecting group;
R¹ is selected from the group consisting of hydrogen, halogen, hydroxy, methoxy, trifluoromethyl, nitro and cyano;
X—Y is selected from the group consisting of —S—CH—, —S—C(CH₃)—, —O—CH—, —O—C(CH₃)—, —N(CH₃)—CH— and —CH═CH—CH—;
A is selected from the group consisting of —CH₂— and —CH(CH₃)—;
R² is hydrogen;
R³ and R⁴ are each independently selected from the group consisting of hydrogen and C₁₋₄alkyl;
alternatively, R³ and R⁴ are taken together with the nitrogen atom to which they are bound to form a 5 to 7 membered, saturated, partially unsaturated or aromatic ring structure, optionally containing one to three additional heteroatoms independently selected from the group consisting of O, N and S.

32. A compound as in claim 31, wherein PG¹ is t-butoxycarbonyl.

33. A compound of formula (XII-S) 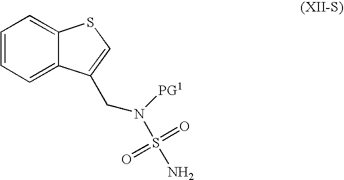
wherein PG¹ is hydrogen or a nitrogen protecting group.

34. A compound as in claim 33, wherein PG¹ is hydrogen or t-butoxycarbonyl.

35. A compound as in claim 33, wherein PG¹ is t-butoxycarbonyl.

36. A compound of formula (XXII) 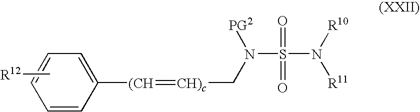
wherein
PG² is hydrogen or a nitrogen protecting group;
R¹² is selected from the group consisting of hydrogen, halogen, hydroxy, methoxy, trifluoromethyl, nitro and cyano;
c is an integer from 0 to 2;
R¹⁰ and R¹¹ are each independently selected from the group consisting of hydrogen and C₁₋₄alkyl;
alternatively, R¹⁰ and R¹¹ are taken together with the nitrogen atom to which they are bound to form a 5 to 7 membered, saturated, partially unsaturated or aromatic ring structure, optionally containing one to three additional heteroatoms independently selected from the group consisting of O, N and S.

37. A compound as in claim 36, wherein R¹² is hydrogen and wherein PG² is hydrogen or a nitrogen protecting group.

38. A compound as in claim 36, wherein R¹² is hydrogen; c is an integer from 0 to 1; R¹⁰ is hydrogen; R¹¹ is hydrogen; and PG² is hydrogen or a nitrogen protecting group.

39. A compound as in claim 38, wherein PG² is t-butoxycarbonyl.
